(19) United States
(12) Patent Application Publication
(10) Pub. No.: US 2004/0096488 A1
(43) Pub. Date: May 20, 2004

(54) Wart treatment adhesive bandage and method of using same

(76) Inventor: Michael Weaver, New Orleans, LA (US)

Correspondence Address: Warner J Delaune Jr, Adams and Reese LLP, 451 Florida Street Bank One Centre, North Tower 19th Floor, Baton Rouge, LA, 70801, US

(21) Appl. No.: 10/298,351

(22) Filed: Nov. 18, 2002

(51) Int. Cl. A61K 9/70; A61L 15/16

(52) U.S. Cl. 424/448

(57) ABSTRACT

An adhesive bandage for the treatment of warts on the human skin is provided, comprising an upper layer of polyethylene coated cloth having a color and texture resembling a conventional bandage and visible upon application to the skin, the upper layer further including a center section and at least one side portion extending from the center section; and a lower layer permanently bonded to and coextending with the center section and the side portion, the lower layer having a rubber-based, waterproof, and nonbreathable adhesive material. The rubber-based adhesive material includes adhesive qualities identical in all respects to that of conventional duct tape. Optionally, the rubber-based adhesive material may be present only directly beneath the center section, while a skin-releasable adhesive material is present only directly beneath the side portion. A method of using the bandage for the treatment of warts is also provided.

### BACKGROUND OF THE INVENTION

I. Field of the Invention

The present invention generally relates to dermatology and treating warts, and more specifically the topical treatment of warts using a polyethylene covered cloth bandage with a natural rubber-based adhesive system.

II. Background and Related Art

A wart develops on the human skin when the Human Papilloma Virus (HPV) infects the outermost layer of skin and causes the skin cells to over-multiply. The following types of warts are found on the human skin: common warts (Verruca vulgaris), plantar warts, palmar warts, planar warts (Verruca plana), mosaic warts, and venereal warts (Condyloma accuminatum). These benign epithelial tumors characterized by the formation of thick hyperkeratotic lesions are irritating and unsightly and their removal is desired.

A variety of therapies have been used in the treatment of warts, including cryotherapy, salicylic acid, cimetidine, cantharidin, podophyllin resin, cryosurgery, carbon dioxide laser, heat and tape occlusion. The current most popular treatment is cryotherapy with liquid nitrogen. A variety of adverse effects with cryotherapy of warts have been previously reported, including pain during the procedure, erythema, hemorrhagic blister formation, dyspigmentation, recurrence of the wart, infection, and nail dystrophy when treating periungual warts. J. M. Plasencia, Cutaneous Warts, Diagnosis, and Treatment, Prim Care, 2000:27:423-434. In addition, cryotherapy can be inconvenient and expensive.

U.S. Pat. No. 5,476,664 entitled “Treatment of warts using anthralins and occlusion” describes one type of occlusion therapy using a composition consisting essentially of an anthralin active ingredient and a pharmaceutical carrier. The invention described below does not use an anthralin active ingredient. Furthermore, the invention described in the '664 patent can require daily applications. Daily application of the wart treatment device can be cumbersome, time consuming and expensive. The duct tape occlusion therapy employed in the invention described below should be reapplied every five to seven days. This makes the invention practical to use and less expensive than daily application.

The wart removing pad described in U.S. Pat. No. 6,471,986 uses salicylic acid on the underside of the pad to treat the wart on the human skin. Salicylic acid is a caustic and locally destructive chemical. The invention described below uses no caustic chemicals in the treatment of warts.

Medical literature contains anecdotal reports of tape occlusion therapy for the treatment of common warts. J. Z. Litt, Don't Exercise-Exorcise: Treatment of Subungual and Periungual Warts, cutis., 1978:22:673-676. According to a report published in the October, 2002 issue of the Archives of Pediatric and Adolescent Medicine, the results of a study conducted at the Madigan Army Medical Center in Tacoma, Wash., indicate that duct tape occlusion therapy exceeded the efficacy of cryotherapy in the treatment of the common wart.

Upon reviewing the references cited above, certain conclusions can be made about the prior art as a whole. First, none of the references discloses the use of the adhesive used in conventional duct tape in the treatment of warts. Second, the prior art treatments of warts which do involve tape occlusion include only materials such as anthralins and the locally destructive chemical salicylic acid. A third conclusion is that duct tape occlusion therapy has resulted in more effective wart treatment than cryotherapy.

Duct tape is a form of adhesive tape used for many years by plumbers in heating, ventilation and air conditioning (HVAC) applications, typically in sealing connections made between adjacent conduits used to deliver heated or cooled air. Because of its inherent flexibility and extremely tacky rubber-based adhesive, duct tape has also been used in a multitude of other household repair situations. Over time, it has become somewhat of a universal tool for eliminating leaks and adhering diverse parts to one another. Coincidentally, duct tape has a number of characteristics which make it appear suitable for the treatment of warts, namely: (1) its non-breathable sealing ability (no respiration from the skin), (2) its extremely sticky undersurface which strongly adheres to the skin, (3) its ability to repel water and other liquids, and (4) its flexibility for use on irregularly shaped surfaces, such as around human appendages.

While duct tape occlusion therapy appears to be effective in the treatment of the common wart, applying silver duct tape to a person's skin can be conspicuous and unattractive. Apart from the appearance, it is quite inconvenient to tear or cut off a suitably sized section of duct tape for use with wart treatment, especially given the unwieldy shape and bulk of a typical duct tape roll. There is a need to provide the effect of duct tape occlusion therapy for the treatment of warts in a design and manner that is attractive, inconspicuous, and convenient. The invention described below achieves this objective by providing a convenient, painless, inexpensive and inconspicuous treatment of warts on the human skin. In effect, the shape and appearance of the present invention is similar to a conventional adhesive bandage, but superior thereto by providing the purported therapeutic effects of duct tape occlusion on the wart.

### SUMMARY OF THE INVENTION

It is an object of the invention to provide an inconspicuous adhesive bandage for treating warts on the human skin.

It is another object of the invention to provide a method for treating warts on the human skin employing an adhesive bandage.

Yet another object of the invention is to provide an adhesive bandage for treating warts on the human skin resembling a conventional bandage.

Still another object of the invention is to provide an adhesive bandage for the treatment of warts on the human skin having effects similar to that attributable to conventional duct tape.

These and other objects and advantages of the invention shall become apparent from the following general and preferred descriptions of the invention.

Accordingly, an adhesive bandage for the treatment of warts on the human skin is provided, comprising an upper layer having a color and texture resembling a conventional bandage and visible upon application to the skin, the upper layer further including a center section and at least one side portion extending from the center section; and a lower layer permanently bonded to and coextending with the center section and the side portion, the lower layer having a natural rubber-based, waterproof, and nonbreathable adhesive material.

In another embodiment, the rubber-based adhesive material may be present only directly beneath the center section, while a skin-releasable adhesive material is present only directly beneath the side portion.

In more preferred embodiments, the rubber-based adhesive material includes adhesive qualities identical in all respects to that of conventional duct tape. Also, the color of the upper layer, preferably constructed from a polyethylene film, is approximately the same as the color of the skin to which the bandage is to be applied, and the upper layer may include a plurality of holes formed therethrough.

In the interest of presenting the appearance of a common bandage, the adhesive bandage may be constructed such that the center section is orthogonal in shape, and wherein a first side portion extends from one side of the center section, and wherein a second side portion extends from an opposite side of the center section. Alternatively, the side portion may comprise an annular portion surrounding the center section.

### BRIEF DESCRIPTION OF THE DRAWINGS

FIG. 1 is a perspective view of a preferred embodiment of the present invention depicting the upper surface of a bandage.

FIG. 2 is a perspective view of the underside of the embodiment of FIG. 1.

FIG. 3 is a detailed cross-sectional view of the embodiment of FIG. 1.

FIG. 4 depicts a bandage of the present invention applied to a wart.

FIG. 5 illustrates an alternative embodiment of the present invention.

### DETAILED DESCRIPTION OF THE INVENTION

In the following detailed description of the preferred embodiments, reference is made to the accompanying drawings which form a part hereof, and in which are shown by way of illustration specific embodiments in which the invention may be practiced. It is to be understood that other embodiments may be utilized and structural changes may be made without departing from the scope of the present invention.

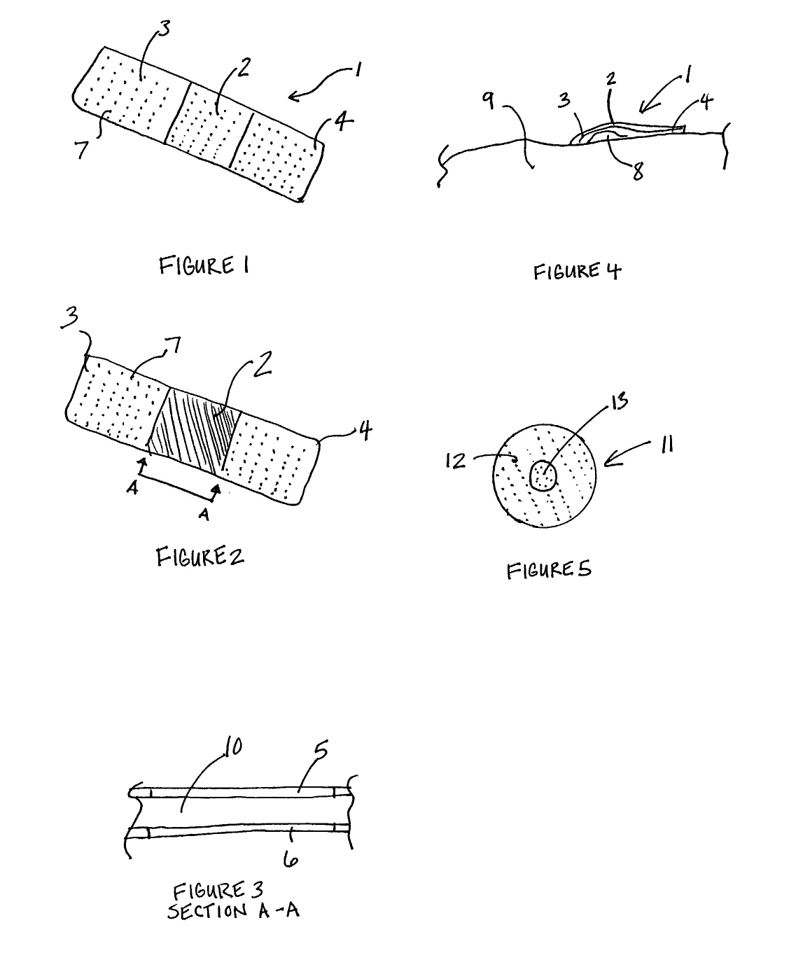

As shown in FIGS. 1-3, a preferred embodiment of the present invention is shown in the form of a bandage 1 generally comprising a center section 2 and opposing side portions 3,4. In conventional bandages known in the prior art, the center section 2 is typically used to cover a wound or skin abrasion, while the side portions 3,4 are adhesively affixed to the skin surrounding the wound. From the ensuing description, it will become clear how the present invention differs from the prior art.

FIG. 1 illustrates an upper layer 5 of the bandage which resembles in all respects a conventional bandage, particularly the visibly distinct center section 2 and the presence of an array of small holes 7 in the center section 2 and the side portions 3,4. Upper layer 5 preferably comprises a relatively thin polyethylene film. In a more preferred embodiment, upper layer 5 also has a flesh-colored appearance so as to blend in with the natural pigmentation of the skin. As depicted in the underside view of FIG. 2 and the cross-sectional view of FIG. 3, the bandage 1 also includes a lower layer 6 having features novel to the present invention. Both upper layer 5 and lower layer 6 are permanently bound to intermediate layer 10, which comprises a fabric material constructed preferably of cotton. Intermediate layer 10 serves as the primary structural member for the bandage 1 and a substrate for adhering upper layer 5 and lower layer 6. The lower layer 6 of side portions 3,4 comprises a skin-releasable adhesive commonly found in conventional bandages in the prior art. By using the term skin-releasable, it is intended that such skin-releasable adhesive permit removal of side portions 3,4 with as little force and pain to the user as possible. As in conventional bandages, and as shown in FIG. 4, the holes 7 pass completely through side portions 3,4 to permit the skin 9 surrounding the wart 8 to breathe through the side portions 3,4. In contrast, although the upper layer 5 of center section 2 includes holes 7, such holes 7 do not penetrate lower layer 6 of center section 2. The lower layer 6 of center section 2 comprises a rubber-based, waterproof, and nonbreathable adhesive substance identical to the adhesive commonly used in conventional duct tape. This adhesive substance has an adhesion of roughly 50 to 55 oz./in. The overall thickness of the bandage ranges from 9 to 14 mils. Therefore, upon applying the bandage 1 to a wart 8, complete coverage of the wart 8 by center section 2 achieves the occlusive, waterproof, and nonbreathable effect desired as when using conventional duct tape. The rubber-based adhesive material employed under center section 2, in contrast to the skin-releasable adhesive under side portions 3,4, is intended to form a strong and sealing bond with the human skin such that removal is not readily accomplished without the exertion of substantial force and perhaps more pain to the user. Such a strong bond is essential to the creation of the required occlusive and waterproof seal around the wart.

FIG. 5 illustrates an alternative embodiment of the present invention in the form of a circular bandage 11. This embodiment is identical in all respects to the preferred embodiment previously described, except that side portions 3,4 are replaced by an annular portion 12 surrounding a center section 13. Annular portion 12 includes the same skin-releasable adhesive as side portions 3,4, while center section 13 includes the same rubber-based adhesive as center section 2. It should be understood to those of ordinary skill in the art that while only the preferred and alternative embodiments are illustrated herein, any shape used in conjunction with conventional bandages may be used for the present invention.

A simpler alternative embodiment of the invention may comprise a single lower layer 6 of rubber-based adhesive extending completely across center section 2 and side portions 3,4. Thus, holes 7 would not extend through side portions 3,4, and this alternative embodiment would not have any breathable portion at all. Depending upon the size of the wart 8 or in the presence of a cluster of warts 8, such an embodiment may actually be preferred, as it would offer a larger area of occluded and waterproof coverage of the affected region. Similarly, with respect to FIG. 5, a single lower layer 6 of rubber-based adhesive may be employed completely across center section 13 and annular portion 12 to achieve an identical result, albeit using a different shape.

A key feature of the invention is the visible appearance of a conventional bandage. Although conventional bandages have a wide variety of surface features, such as the specific pattern of holes 7 or texture, the color of the surface, artistic images, and even cartoon characters, the bandage 1 of the present invention may include any of such features. This is particularly important because it minimizes the appearance of the bandage on the skin while achieving the same benefits of treatment of the wart using duct tape. As is common with conventional bandages, the bandages 1,11 of any of the aforedescribed embodiments may include removable protective layers (not shown) covering the adhesive regions of lower layer 6 during storage of the bandages, and which can be easily removed immediately prior to actual application onto the skin.

In application of the present invention to therapeutic use, the bandage 1,11 is applied to the wart 8 such that the rubber-based adhesive of lower layer 6 completely covers the wart 8 and an area of said skin immediately surrounding the wart 8. In doing so, a waterproof and occlusive seal is thereby established around the wart 8. Next, the bandage 1,11 is allowed to remain on the wart 8 for a predetermined number of consecutive days, typically from five to seven consecutive days. Once the initial treatment period is over, the bandage 1,11 is removed from the wart 8, and the wart 8 is soaked in warm water to soften the calloused or hardened skin. Next, an emory board, pumice stone, or other suitable abrasive material is used to rub or abrade the wart, thus removing much of the dead skin created by application of the bandage 1,11. The bandage is left off overnight, typically 6 to 10 hours, and reapplied the following morning. To recommence the treatment process on that portion of the wart 8 remaining on the skin 9. The process described herein can be repeated until the entire wart 8 is removed.

As will be appreciated, the foregoing embodiments of the present invention satisfy the objectives of convenience and attractiveness for what it an otherwise unattractive skin condition. Although the present invention has been described in terms of specific embodiments, it is anticipated that alterations and modifications thereof will no doubt become apparent to those skilled in the art. It is therefore intended that the following claims be interpreted as covering all alterations and modifications that fall within the true spirit and scope of the invention.

What is claimed is:

1. An adhesive bandage for the treatment of warts on the human skin, comprising:
an upper layer having a color and texture resembling a conventional bandage and visible upon application to said skin, said upper layer further including a center section and at least one side portion extending from said center section; and
a lower layer permanently bonded to and coextending with said center section and said side portion, said lower layer having a rubber-based, waterproof, and nonbreathable adhesive material.

2. The adhesive bandage of claim 1, wherein said adhesive material includes adhesive qualities identical in all respects to that of duct tape.

3. The adhesive bandage of claim 1, wherein said color is approximately the same as the color of said skin to which said bandage is to be applied.

4. The adhesive bandage of claim 1, wherein said upper layer includes a plurality of holes formed therethrough.

5. The adhesive bandage of claim 1, wherein said upper layer comprises a polyethylene film.

6. The adhesive bandage of claim 1, wherein said center section is orthogonal in shape, and wherein a first side portion extends from one side of said center section, and wherein a second side portion extends from an opposite side of said center section.

7. The adhesive bandage of claim 1, wherein said side portion comprises an annular portion surrounding said center section.

8. An adhesive bandage for the treatment of warts on the human skin, comprising:
an upper layer having a color and texture resembling a conventional bandage and visible upon application to said skin, said upper layer further including a center section and at least one side portion extending from said center section; and
a lower layer permanently bonded to and coextending with said center section and said side portion, said lower layer having:
(a) a rubber-based, waterproof, and nonbreathable adhesive material directly beneath and confined to the area defined by said center section for contacting said skin; and
(b) a skin-releasable adhesive material directly beneath and confined to the area defined by said side portion for contacting said skin.

9. The adhesive bandage of claim 8, wherein said rubber-based adhesive material includes adhesive qualities identical in all respects to that of duct tape.

10. The adhesive bandage of claim 8, wherein said color is approximately the same as the color of said skin to which said bandage is to be applied.

11. The adhesive bandage of claim 8, wherein said upper layer includes a plurality of holes formed therethrough.

12. The adhesive bandage of claim 8, wherein said upper layer comprises a polyethylene film.

13. The adhesive bandage of claim 8, wherein said center section is orthogonal in shape, and wherein a first side portion extends from one side of said center section, and wherein a second side portion extends from an opposite side of said center section.

14. The adhesive bandage of claim 8, wherein said side portion comprises an annular portion surrounding said center section.

15. A method of treating warts on the human skin, comprising the steps of:
providing an adhesive bandage comprising:
(a) an upper layer having a color and texture resembling a conventional bandage and visible upon application to said skin, said upper layer further including a center section and at least one side portion extending from said center section; and
(b) a lower layer permanently bonded to and coextending with said center section and said side portion, said lower layer having a rubber-based, waterproof, and nonbreathable adhesive material for contacting said skin;
applying said bandage to said wart such that said rubber-based adhesive completely covers said wart and an area of said skin immediately surrounding said wart, thereby creating a waterproof and occlusive seal around said wart;
allowing said bandage to remain on said wart for a predetermined number of consecutive days;
removing said bandage from said wart;
soaking said wart in warm water;
rubbing said wart with an abrasive material;
providing a non-treatment period during which said bandage is not applied to said wart for a predetermined number of hours to permit said wart to dry; and
reapplying said bandage to said wart and repeating the previous steps until said wart is removed.
